(19) United States
(12) Patent Application Publication
(10) Pub. No.: US 2025/0375311 A1
(43) Pub. Date: Dec. 11, 2025

(54) BRACE-TYPE ANTI-SNORING DEVICE

(71) Applicant: PIETRO CANDRIELLA, Dubai (AE)

(72) Inventor: PIETRO CANDRIELLA, Dubai (AE)

(21) Appl. No.: 18/789,191

(22) Filed: Jul. 30, 2024

(30) Foreign Application Priority Data
Jun. 11, 2024 (CN) 202421320948.0

(51) Int. Cl. A61F 5/56

(52) U.S. Cl. CPC A61F 5/566

(57) ABSTRACT

The disclosure belongs to the technical field of snoring prevention, in particular to a brace-type anti-snoring device, which includes an upper brace, a lower brace and a brace mounting seat. The upper brace and the lower brace are both fixedly arranged on the brace mounting seat. An upper surface of the upper brace is provided with an upper tooth socket, and the upper tooth socket is configured for embedding teeth of an upper jaw; and a lower surface of the lower brace is provided with a lower tooth socket, and the lower tooth socket is configured for embedding teeth on a lower jaw and restricting the lower jaw from moving backwards.

### TECHNICAL FIELD

The disclosure belongs to the technical field of snoring prevention, in particular to a brace-type anti-snoring device.

### BACKGROUND ART

Snoring mainly results from increase in respiratory resistance caused by obstruction or stenosis of respiratory tract during sleep, which further hinders an air flow through the respiratory tract and causes soft tissue of a throat to vibrate and make noise, that is, snoring. However, for some people, one of reasons for the increase in respiratory resistance during sleep is that the human mandible can move forward and backward, and during sleep in a lying manner, the mandible may move backward under action of gravity, causing a tongue in a relaxed state and other muscle tissues to block the trachea, which in turn results in a problem of snoring.

Therefore, there's a need to design a brace-type anti-snoring device that can prevent the mandible from moving backwards during sleep in the lying manner.

### SUMMARY

In order to solve above problems existing in related art, a brace-type anti-snoring device is provided in the disclosure. The technical scheme adopted in the disclosure is as follows.

A brace-type anti-snoring device includes an upper brace, a lower brace and a brace mounting seat. The upper brace and the lower brace are both fixedly arranged on the brace mounting seat. An upper surface of the upper brace is provided with an upper tooth socket, and the upper tooth socket is configured for embedding teeth of an upper jaw; and a lower surface of the lower brace is provided with a lower tooth socket, and the lower tooth socket is configured for embedding teeth on a lower jaw and restricting the lower jaw from moving backwards.

As an alternative or in addition to the brace-type anti-snoring device, the upper brace and/or the lower brace are detachably connected with the brace mounting seat in a buckling mode, a screwing mode or a snapping mode.

As an alternative or in addition to the brace-type anti-snoring device, the upper brace and/or the lower brace are buckled with the brace mounting seat.

As an alternative or in addition to the brace-type anti-snoring device, the lower surface of the upper brace is provided with one or more upper protruding buckles, and the brace mounting seat is defined with first clamping holes correspondingly connected with the upper protruding buckles.

As an alternative or in addition to the brace-type anti-snoring device, the upper surface of lower facing is provided with one or more lower protruding buckles, and the brace mounting seat is further defined with second clamping holes correspondingly connected with the lower protruding buckles.

As an alternative or in addition to the brace-type anti-snoring device, the first clamping holes and the second clamping holes are elongated in shape, a length direction for the first clamping holes is same as a front-back direction of the upper brace, and a length direction for the second clamping holes is same as a front-back direction of the lower brace.

As an alternative or in addition to the brace-type anti-snoring device, the first clamping holes and the second clamping holes both present a plurality of clamping positions arranged in a straight line, a front-back position of the upper brace varies when the upper protruding buckles are connected to different clamping positions of the first clamping hole, and a front-back position of the lower brace varies when the lower protruding buckles are connected to different clamping positions of the second clamping holes.

The lower protruding buckles and the upper protruding buckles are both of a double-prism structure, and the double-prism structure is a structure formed by intersecting edges of two quadrangular prisms.

As an alternative or in addition to the brace-type anti-snoring device, the brace mounting seat includes an upper connecting part, a U-shaped connecting part and a lower connecting part. The upper connecting part is arranged above the lower connecting part, and two ends of the U-shaped connecting part are connected with the upper connecting part and the lower connecting part respectively.

As an alternative or in addition to the brace-type anti-snoring device, the upper connecting part and the lower connecting part are both U-shaped, and two ends of the upper connecting part are correspondingly connected with two ends of the lower connecting part through the U-shaped connecting part.

As an alternative or in addition to the brace-type anti-snoring device, an inner side edge of the lower tooth socket exceeds an outer side edge of the lower tooth socket.

As an alternative or in addition to the brace-type anti-snoring device, the upper connecting part and the lower connecting part are of a flat block structure. The disclosure has following advantages.

1. In the brace-type anti-snoring device according to the disclosure, the upper jaw can be fixed in use and the lower jaw is limited from moving backwards, so that the mandible can be prevented from moving backwards in a lying state to cause a tongue in a relaxed state and other muscle tissues to block the trachea.

2. In addition, the upper brace, the lower brace and the brace mounting seat in the disclosure can adopt a detachable connection structure, so that the upper brace, the lower brace and the brace mounting seat can be facilitated to be detached from one another and replaced so as to better match shapes of teeth of different persons; and universality and the popularity are improved.

3. In the disclosure, the brace mounting seat composed of the upper connecting part, the U-shaped connecting part and the lower connecting part is adopted, which causes the upper brace and the lower brace to be more closely attached to a human mouth and users to freely open and close their mouths.

### BRIEF DESCRIPTION OF THE DRAWINGS

In order to more clearly explain technical schemes in embodiments of this disclosure or the related art, drawings needed in description of the embodiments or the related art will be briefly introduced below.

FIG. 1 is an explosive structural view of a brace-type anti-snoring device according to the disclosure;

FIG. 2 is a schematic structural view of a brace mounting seat according to the disclosure;

FIG. 3 is a perspective structural view of a brace-type anti-snoring device according to the disclosure;

FIG. 4 is a three-dimensional structural view of a brace-type anti-snoring device according to the disclosure from in a bottom view; and

FIG. 5 is a side structural view of a brace-type anti-snoring device according to the disclosure.

Reference numbers are as follows: 1—Lower Brace; 11—Lower Protruding Buckle; 12—Lower Tooth Socket; 13—Front Edge; 14—Back Edge; 2—Brace Mounting Seat; 21—Upper Connecting Part; 22—U-shaped Connecting Part; 23—First Clamping Hole; 24—Lower Connecting Part; 3—Upper Brace; 31—Upper Protruding Buckle; 32—Upper Tooth Socket.

### DETAILED DESCRIPTION

The technical schemes in this disclosure will be described clearly and completely in combination with the drawings. The described embodiments are only a part of embodiments, but not all of them. On a basis of the embodiments in this disclosure, all other embodiments obtained by the ordinary skilled in the art without any creative effort are within the protection scope of this disclosure.

### Embodiment 1

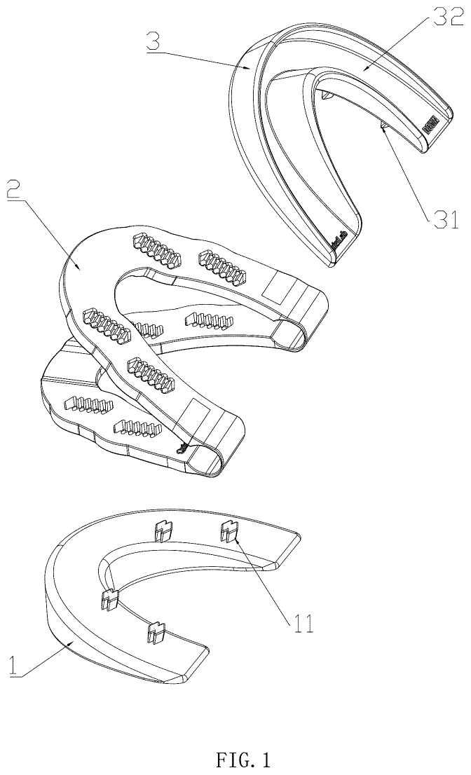

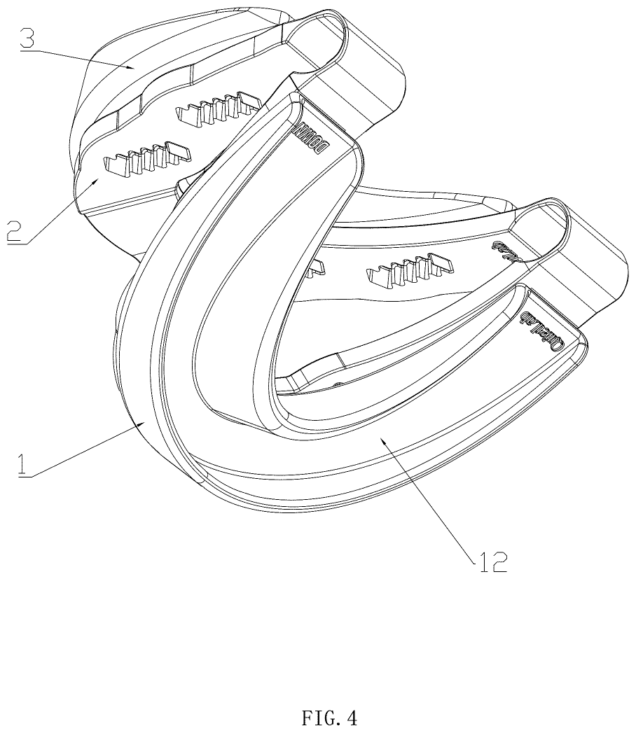

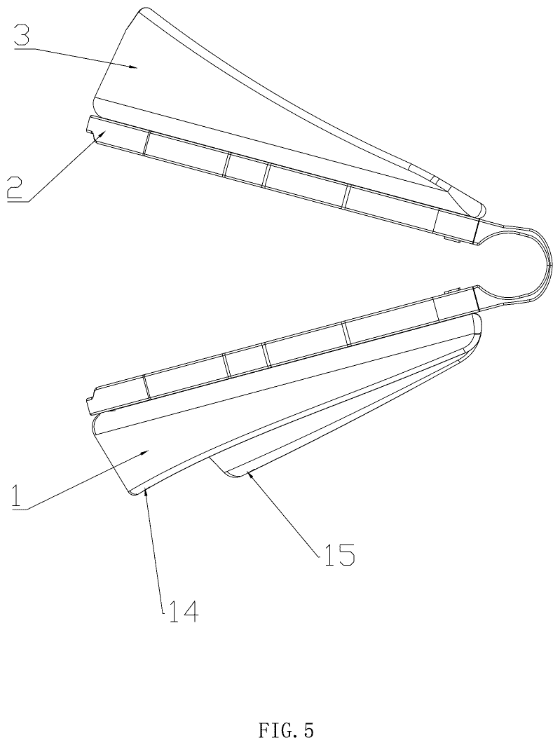

As shown in FIGS. 1 to 5, a brace-type anti-snoring device is designed in the disclosure, which includes an upper brace 3, a lower brace 1 and a brace mounting seat 2.

The upper brace 3 and the lower brace 1 are both fixedly arranged on the brace mounting seat 2. The upper brace 3 and/or the lower brace 1 can be fixedly connected with the brace mounting seat 2. The upper brace 3 and/or the lower brace 1 are detachably connected with the brace mounting seat 2 in a buckling mode, a screwing mode or a snapping mode, so that the upper brace, the lower brace and the brace mounting seat can be facilitated to be detached from one another and replaced so as to better match shapes of teeth of different persons; and universality and the popularity are improved.

An upper surface of the upper brace 3 is provided with an upper tooth socket 32, and the upper tooth socket 32 is configured for embedding teeth of an upper jaw; and when the upper brace 3 is mounted on the teeth of the upper jaw, a position of the brace-type anti-snoring device can be fixed, thus providing basis for the brace-type anti-snoring device to support the lower jaw. A height of a front side of the upper brace 3 can be 13.415 mm±2 mm, and the height of the upper brace 3 decreases from front to back.

A lower surface of the lower brace 1 is provided with a lower tooth socket 12, and the lower tooth socket 12 is configured for embedding teeth on a lower jaw. When the teeth of the lower jaw bite on the lower brace 1, the lower brace 1 can support the lower jaw, so as to limit a front-back position of the lower jaw relative to the upper jaw, limit backward movement of the lower jaw, so that the mandible can be prevented from moving backwards in a lying state to cause a tongue in a relaxed state and other muscle tissues to block the trachea.

When the upper brace 3 and the lower brace 1 are connected with the brace mounting base 2 in the buckling mode, the lower surface of the upper brace 3 is provided with one or more upper protruding buckles 31, and the brace mounting seat 2 is defined with first clamping holes 23 correspondingly connected with the upper protruding buckles 31. The upper surface of lower facing 1 is provided with one or more lower protruding buckles 11, and the brace mounting seat 2 is further defined with second clamping holes correspondingly connected with the lower protruding buckles 11.

As another embodiment of the disclosure, the first clamping holes 23 and the second clamping holes both present a plurality of clamping positions arranged in a straight line, a front-back position of the upper brace 3 varies when the upper protruding buckles 31 are connected to different clamping positions of the first clamping hole 23, and a front-back position of the lower brace 1 varies when the lower protruding buckles 11 are connected to different clamping positions of the second clamping holes. Both the upper protruding buckles 31 and the lower protruding buckles 11 are matched with corresponding clamping positions in shape, and when the upper protruding buckles 31 are in a quadrangular prism shape, the clamping positions are also in a form of a quadrangular prism shaped hole. The first clamping holes 23 and the second clamping holes may be in a structure of an elongated hole formed by intersecting a plurality of quadrangular holes. The clamping positions are arranged in a front-back direction. Specifically, a length direction for the first clamping holes 23 is same as a front-back direction of the upper brace 3, and a length direction for the second clamping holes is same as a front-back direction of the lower brace 1.

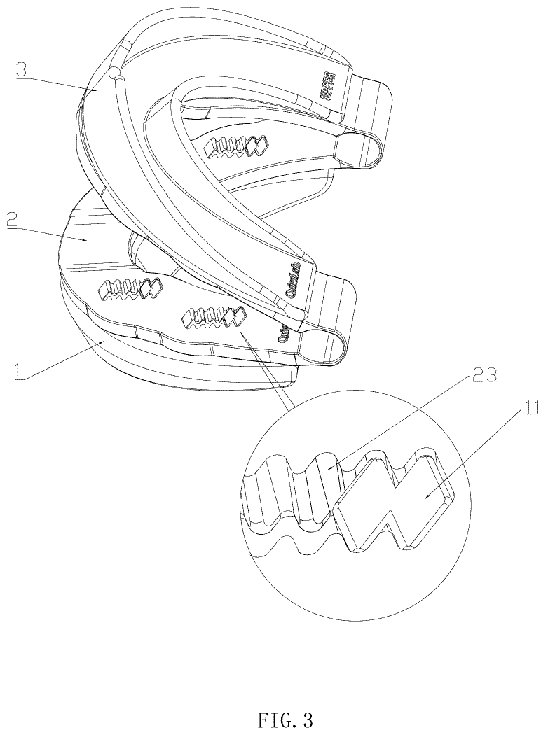

Both the lower protruding buckles 11 and the upper protruding buckles 31 adopt a double-prism structure, and the double-prism structure is a structure formed by intersecting edges of two quadrangular prisms, which can be detailed in FIG. 3.

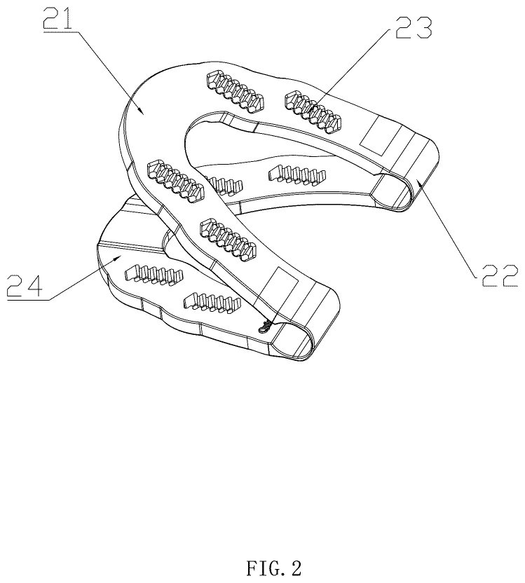

The brace mounting seat 2 can adopt a flat block-shaped structure, and the upper brace 3 and the lower brace 1 are respectively fixed to an upper surface and a lower surface of the brace mounting seat 2. Alternatively, the brace mounting seat 2 may also include components such as an upper connecting part 21, a U-shaped connecting part 22 and a lower connecting part 24, as shown in FIG. 2. The upper connecting part 21 is arranged above the lower connecting part 24, and two ends of the U-shaped connecting part 22 are connected with the upper connecting part 21 and the lower connecting part 24 respectively. The upper connecting part 21 and the lower connecting part 24 are both U-shaped, and two ends of the upper connecting part 21 are correspondingly connected with two ends of the lower connecting part 24 through the U-shaped connecting part 22.

The upper connecting part 21 and the lower connecting part 24 are of a flat block structure. After the upper brace 3 and the lower brace 1 are connected with the brace mounting seat 2, the lower surface of the upper brace 3 is attached to the upper connecting part 21, and the upper surface of the lower brace 1 is attached to the lower connecting part 24.

The upper brace 3 and the lower brace 1 can be made of a TPE silicone material with hardness of 60 HB; and the brace mounting seats 2 can also be made of the TPE silicone material with a hardness of 90 HB.

The above embodiments are only for clearly explaining examples made, and are not for limiting implementations. It is not necessary and impossible to exhaust all the embodiments herein. Obvious changes or variations derived are still within the protection scope of this disclosure.

1. A brace-type anti-snoring device, comprising an upper brace (3), a lower brace (1) and a brace mounting seat (2), wherein the upper brace (3) and the lower brace (1) are both fixedly arranged on the brace mounting seat (2), an upper surface of the upper brace (3) is provided with an upper tooth socket (32), and the upper tooth socket (32) is configured for embedding teeth of an upper jaw; and a lower surface of the lower brace (1) is provided with a lower tooth socket (12), and the lower tooth socket (12) is configured for embedding teeth on a lower jaw and restricting the lower jaw from moving backwards;
the upper brace (3) and/or the lower brace (1) are detachably connected with the brace mounting seat (2) in a buckling mode, a screwing mode or a snapping mode;
the lower surface of the upper brace (3) is provided with one or more upper protruding buckles (31), and the brace mounting seat is (2) defined with first clamping holes (23) correspondingly connected with the upper protruding buckles (31); and
the upper surface of lower facing (1) is provided with one or more lower protruding buckles (11), and the brace mounting seat (2) is further defined with second clamping holes correspondingly connected with the lower protruding buckles (11).

2. The brace-type anti-snoring device according to claim 1, wherein the first clamping holes (23) and the second clamping holes are elongated in shape, a length direction for the first clamping holes (23) is same as a front-back direction of the upper brace (3), and a length direction for the second clamping holes is same as a front-back direction of the lower brace (1).

3. The brace-type anti-snoring device according to claim 2, wherein the first clamping holes (23) and the second clamping holes both present a plurality of clamping positions arranged in a straight line, a front-back position of the upper brace (3) varies when the upper protruding buckles (31) are connected to different clamping positions of the first clamping hole (23), and a front-back position of the lower brace (1) varies when the lower protruding buckles (11) are connected to different clamping positions of the second clamping holes.

4. The brace-type anti-snoring device according to claim 3, wherein the lower protruding buckles (11) and the upper protruding buckles (31) are both of a double-prism structure, and the double-prism structure is a structure formed by intersecting edges of two quadrangular prisms.

5. The brace-type anti-snoring device according to claim 1, wherein the brace mounting seat (2) comprises an upper connecting part (21), a U-shaped connecting part (22) and a lower connecting part (24), the upper connecting part (21) being arranged above the lower connecting part (24), and two ends of the U-shaped connecting part (22) being connected with the upper connecting part (21) and the lower connecting part (24) respectively.

6. The brace-type anti-snoring device according to claim 5, wherein the upper connecting part (21) and the lower connecting part (24) are both U-shaped, and two ends of the upper connecting part (21) are correspondingly connected with two ends of the lower connecting part (24) through the U-shaped connecting part (22).

7. The brace-type anti-snoring device according to claim 6, wherein an inner side edge of the lower tooth socket (12) exceeds an outer side edge of the lower tooth socket.
